(19) United States
(12) Patent Application Publication
(10) Pub. No.: US 2014/0255968 A1
(43) Pub. Date: Sep. 11, 2014

(54) METHOD FOR FABRICATING A PATTERNED SUBSTRATE FOR A CELL CULTURE, A PATTERNED SUBSTRATE FOR CELL CULTURE, AND A CELL CHIP

(71) Applicant: RESEARCH & BUSINESS FOUNDATION SUNGKYUNKWAN UNIVERSITY, Suwon-si (KR)

(72) Inventors: Dong Geun JUNG, Seoul (KR); Myung Hoon HA, Suwon-si (KR); Heon Yong PARK, Yongin-si (KR); Ji Soo PARK, Paju-si (KR); Hye Rim LEE, Suwon-si (KR)

(73) Assignee: RESEARCH & BUSINESS FOUNDATION SUNGKYUNKWAN UNIVERSITY, Suwon-si (KR)

(21) Appl. No.: 14/183,961

(22) Filed: Feb. 19, 2014

(30) Foreign Application Priority Data
Mar. 8, 2013 (KR) 10-2013-0025350

(51) Int. Cl. C12N 5/00; G01N 33/50

(52) U.S. Cl. CPC C12N 5/0068; G01N 33/5008; G01N 2500/04
USPC 435/29; 435/396; 435/289.1; 435/420; 435/243; 427/488; 427/491

(57) ABSTRACT

The present invention relates to a method for fabricating a patterned substrate for a cell culture, comprising the steps of: (1) preparing a substrate; (2) depositing a plasma polymer layer by using a precursor material on the substrate; (3) placing a shadow mask having a predetermined pattern on the plasma polymer layer; (4) treating the substrate, having the shadow mask placed thereon, with a reactive gas using plasma; and (5) removing the shadow mask from the substrate, and a patterned substrate for the cell culture fabricated thereby. The invention also relates to a method for a cell culture with a pattern, comprising the step of culturing cells on the patterned substrate for the cell culture, and a patterned cell chip, and a method of screening a material having an activity of inducing or promoting angiogenesis using the patterned cell chip.

### CROSS-REFERENCE TO RELATED APPLICATIONS

This application claims the benefit under 35 USC 119(a) of Korean Patent Application No. 10-2013-0025350 filed on Mar. 8, 2013 in the Korean Intellectual Property Office, the entire disclosure of which is incorporated herein by reference for all purposes.

### BACKGROUND

1. Field

The present invention relates to a method for fabricating a patterned substrate for a cell culture, a patterned substrate for the cell culture fabricated thereby, a method for a cell culture with a pattern, a patterned cell chip, and a method of screening a material having an activity of inducing or promoting angiogenesis using the same.

2. Description of Related Art

Biochips are a kind of chip devices made from a combination of organic biomolecules, such as biological enzymes, proteins, antibodies, DNAs, microorganisms, animal/plant cells and organs, nerve cells and organs, and inorganic materials such as glass. Biochips can be largely classified into “DNA chips” having DNA probes immobilized thereon, “protein chips” having proteins immobilized thereon such as enzymes, antibodies or antigens, and “cell chips” having cells immobilized thereon.

Among them, a cell chip capable of culturing a large amount of cells without changing their properties is an effective tool that can be applied to various fields, including new drug development, genomics and proteomics. The cell chip differs somewhat from a protein chip in that the growth rate of cells on the substrate of the cell chip is also an index indicating the performance of the cell chip. When cells can be cultured on a substrate to grow and divide, the cells can be easily analyzed. Thus, the cell chip has an advantage in that, for example, the effect of new drugs on cells or the response of cells to biological substances such as hormones can be easily examined.

Various methods for culturing cells on substrates are known. These methods can be largely classified into methods utilizing biomaterials, and methods of culturing cells using the physical and chemical properties of substrates. The methods utilizing biomaterials include a method that comprises immobilizing biomaterials such as peptides or proteins on a substrate, and then culturing cells using the cell receptors of these biomaterials [Mann B K et al. Modification of surfaces with cell adhesion peptides alters extracellular matrix deposition. Biomaterials, 1999, 20(23-24): 2281-2286].

In addition, the methods utilizing the physical and chemical properties of substrates include a method utilizing the hydrophobic properties of a substrate, a method utilizing the electrical properties of a substrate, a method utilizing the surface structures of a substrate [Curtis A S et al. Reactions of cells to topography. J. Biomater. Sci.-Polym. Ed., 1998, 9(12): 1313-1329], a method of culturing cells using collagen [On-chip transfection of PC12 cells based on the rational understanding of the role of ECM molecules: efficient, non-viral transfection of PC12 cells using collagen IV. Neuroscience Letters, 2005, 378(1): 40-43], etc.

To develop such biochips, it is important to develop a biomaterial immobilization technology that can efficiently form an interface between biomaterials and a substrate and enable the intrinsic functions of biomaterials to be maximally used.

With respect to this technology, International Patent Publication No. WO 2008/001117 discloses a method for fabricating a cell culture substrate, a cell culture substrate, a method for immobilizing cells, and a cell chip. This patent publication relates to a method for fabricating a cell culture substrate for efficiently culturing cells, which comprises depositing a large amount of functional groups on a substrate using plasma, a cell culture substrate, a cell culture method, and a cell chip. The above-mentioned patent publication merely discloses the method of immobilizing cells on the substrate using plasma, but does not describe a method of selectively culturing cells using the adsorption and inhibition of adsorption of cells.

When a substrate is patterned to have a surface that inhibits the adsorption of cells and a surface on which cells can be easily cultured, it can be applied for the development of chips for insertion into the human body, the development of artificial organs, and genetic experiments, drug tests and the like, which use cells. However, the above-described patent publication merely discloses the method of uniformly culturing cells, and thus it is impossible to selectively culture a small amount of cells in a desired position.

In addition, Korean Patent Laid-Open Publication No. 2011-0024244 discloses a method for fabricating a patterned substrate for cell culture, a patterned substrate for cell culture, and a cell chip. According to the disclosure of this patent publication, the cell culture substrate is fabricated by a method of immobilizing precursor-derived materials on the substrate using plasma. This method has shortcomings in that two or more precursor materials are required and two or more deposition processes should be performed, and thus the patterning process is complicated.

Accordingly, the present inventors have made extensive efforts to develop a patterned substrate for cell culture by a simpler fabrication method, and as a result, have found that, when a substrate is treated with a reactive gas using plasma, it is possible to pattern the substrate in a simple manner so as to enable to culture cells selectively in a desired area of the substrate, and fabricate a patterned substrate for cell culture and a cell chip using the simple patterning technology, thereby completing the present invention.

### SUMMARY

An object of the present invention is to provide a method for fabricating a patterned substrate for a cell culture, comprising the steps of: (1) preparing a substrate; (2) depositing a plasma polymer layer by using a precursor material on the substrate; (3) placing a shadow mask having a predetermined pattern on the plasma polymer layer; (4) treating the substrate, having the shadow mask placed thereon, with a reactive gas using plasma; and (5) removing the shadow mask from the substrate, and a patterned substrate for a cell culture fabricated by the above method.

Another object of the present invention is to provide a method for a cell culture with a pattern, comprising the step of culturing cells on the patterned substrate for the cell culture fabricated by the above method.

A further object of the present invention is to provide a patterned cell chip having cells cultured on the patterned substrate for cell culture fabricated by the above method.

A further object of the present invention is to provide a method of screening a material having an activity of inducing or promoting angiogenesis, using the patterned cell chip.

In order to accomplish the above objects, an aspect of the present invention provides a method for fabricating a patterned substrate for a cell culture, comprising the steps of: (1) preparing a substrate; (2) depositing a plasma polymer layer by using a precursor material on the substrate; (3) placing a shadow mask having a predetermined pattern on the plasma polymer layer; (4) treating the substrate, having the shadow mask placed thereon, with a reactive gas using plasma; and (5) removing the shadow mask from the substrate.

Another aspect of the present invention provides a patterned substrate for a cell culture fabricated by the above method.

A further aspect of the present invention provides a method for a cell culture with a pattern, comprising the step of culturing cells on the patterned substrate for the cell culture fabricated by the above method.

A further aspect of the present invention provides a patterned cell chip having cells cultured on the patterned substrate for cell culture fabricated by the above method.

A further aspect of the present invention provides a method of screening a material having an activity of inducing or promoting angiogenesis, using the patterned cell chip.

### BRIEF DESCRIPTION OF THE DRAWINGS

FIG. 1 is a schematic drawing illustrating a method for fabricating a patterned substrate for a cell culture according to the present invention.

FIG. 2 schematically shows a device for plasma enhanced chemical vapor deposition that is used to form a plasma polymer layer on a substrate.

FIG. 3 schematically shows a device for inductively coupled chemical vapor deposition that is used to treat a substrate with a reactive gas.

FIG. 4 shows cells that are cultured while forming a pattern on a patterned substrate for a cell culture fabricated according to an example of the present invention.

FIG. 5 is a schematic drawing illustrating the pattern of a patterned substrate for a cell culture fabricated according to an example of the present invention.

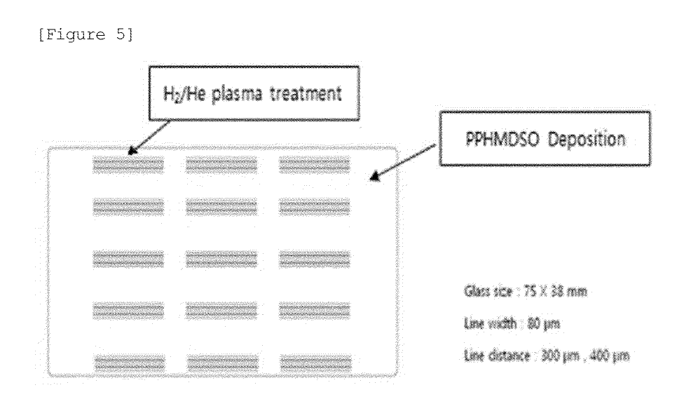

FIG. 6 shows the results of performing an angiogenesis assay on a patterned cell chip of the present invention in an untreated state, a VEGF-treated state and an LPS-treated state.

FIG. 7 is a graphic diagram showing the results of quantification of angiogenesis on a patterned cell chip of the present invention in an untreated state, a VEGF-treated state and an LPS-treated state.

### DETAILED DESCRIPTION

The present invention provides a method for fabricating a patterned substrate for a cell culture, comprising the steps of: (1) preparing a substrate; (2) depositing a plasma polymer layer by using a precursor material on the substrate; (3) placing a shadow mask having a predetermined pattern on the plasma polymer layer; (4) treating the substrate, having the shadow mask placed thereon, with a reactive gas using plasma; and (5) removing the shadow mask from the substrate.

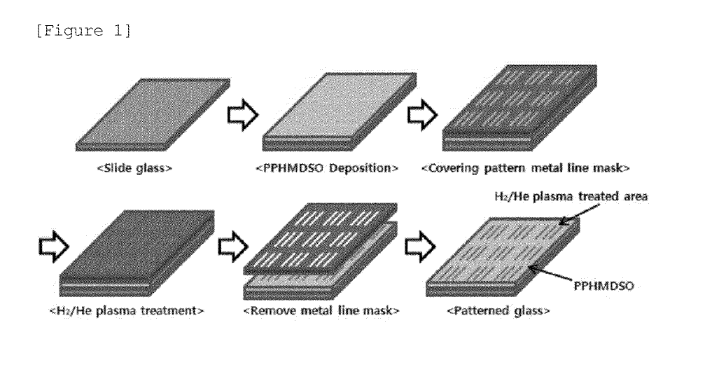

FIG. 1 schematically shows a method for fabricating a patterned substrate for a cell culture according to the present invention. Hereinafter, each step of the fabrication method will be described in detail with reference to FIG. 1.

(1) A Step of Preparing a Substrate

As used herein, the term “substrate” refers to any kind of plate on which a precursor-derived material can be deposited using plasma. Specifically, the substrate may be made of a material such as glass, plastic, metal or silicon, and the kind of substrate is not specifically limited, as long as a precursor-derived material can be deposited thereon using plasma. Preferably, the substrate may be a glass slide.

(2) A Step of Depositing a Plasma Polymer Layer by Using a Precursor Material on the Substrate

As used herein, the term “plasma” refers to the state of electrically neutral gaseous molecules separated into ions and electrons by absorption of electrical energy or thermal energy. Currently, studies on technologies using plasma are being actively conducted, and the scope of applications thereof are expanding to include plasma etching and plasma enhanced chemical vapor deposition (PE-CVD), which are used in semiconductor manufacturing processes, surface treatment of metals or polymers, synthesis of new materials such as synthetic diamond, plasma display panels (PDPs), and environmental purification technologies.

As used herein, the term “precursor material” means a preceding material capable of forming a plasma polymer layer using plasma.

The precursor material that is used in the present invention is not specifically limited, as long as it can inhibit the adsorption of cells. Preferably, the precursor material may be a siloxane-based material such as hexamethyldisiloxane, octamethyltrisiloxane, decamethyltetrasiloxane, hexamethylcyclotrisiloxane, octamethyl cyclotetrasiloxane or decamethylcyclopentasiloxane. More preferably, the precursor material may be hexamethyldisiloxane.

The plasma polymer layer formed by using the precursor material has an excellent advantage in terms of inhibiting the adsorption of cells, because it inhibits the binding between the substrate and cells.

As used herein, the term “deposition” means decomposing a precursor material by plasma energy and forming a plasma polymer layer from the decomposition product.

The plasma polymer layer can be formed by plasma enhanced chemical vapor deposition (PE-CVD).

Plasma enhanced chemical vapor deposition (PE-CVD) is a kind of chemical vapor deposition (CVD) that is often used to form a thin layer like an active layer, an insulating layer and a protective layer on a substrate such as a silicon wafer or glass by chemical reactions during the manufacture of semiconductor devices or flat panel display devices. Specifically, plasma enhanced chemical vapor deposition is a method in which a gas containing a specific chemical compound is excited into a plasma state by radiofrequency power so that the chemical compound contained in the gas can become a radical and/or significantly enhanced in its reactivity, thereby adsorbed and deposited onto the substrate.

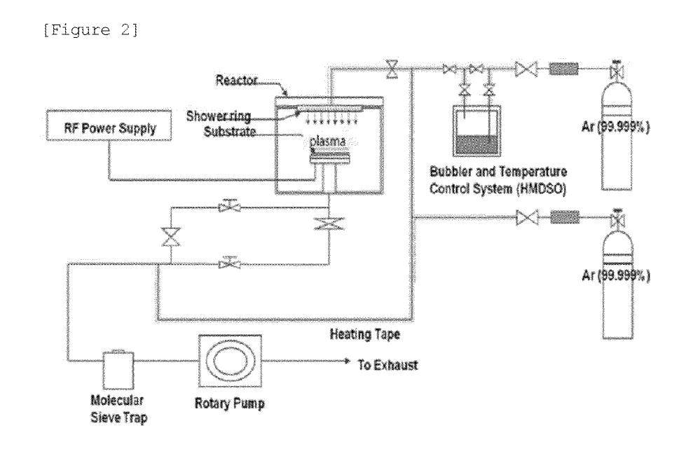

A device for plasma enhanced chemical vapor deposition for performing the step (2) will now be described in detail with reference to FIG. 2. FIG. 2 schematically shows a device for plasma enhanced chemical vapor deposition that is used to form a plasma polymer layer on a substrate.

The device for plasma enhanced chemical vapor deposition shown in FIG. 2 comprises: a plasma reactor configured to form plasma therein; a vacuum unit including a vacuum pump configured to control the internal pressure of the plasma reactor; a gas injection unit including a bubbler and configured to inject a gaseous precursor material into the plasma reactor; and a radio-frequency (RF) power supply unit configured to apply voltage to an internal electrode provided in the plasma reactor. The plasma reactor includes a substrate holder and an internal electrode disposed beneath the substrate holder to support the substrate holder in sequential.

The step of depositing the plasma polymer layer by using the precursor material on the substrate with the system for plasma enhanced chemical vapor deposition will now be described in detail.

First, the substrate is placed on the substrate holder in the plasma reactor. Then, the internal pressure of the plasma reactor is lowered to several mTorr close to a vacuum state by the vacuum pump (rotary pump), and in this state, a precursor material together with a carrier gas is injected into the plasma reactor through the gas injection unit. When a voltage is applied to the internal electrode from the power supply unit in this state, plasma generated around the internal electrode is formed between the substrate and the inner wall of the plasma reactor. At this time, the precursor material is polymerized by the generated plasma while a plasma polymer layer is formed uniformly on the substrate.

Both the external electrode and the internal electrode may be used, but the use of the internal electrode alone is more efficient and effective for formation of the plasma polymer layer. In addition, power that is applied to the internal electrode as a power from the power supply unit of the plasma reactor may be 5-20 W, and preferably 10 W.

Moreover, the precursor material is preferably vaporized before being injected into the plasma reactor. The vaporization of the precursor material is preferably performed by using a method for vaporization in which the precursor material evaporates by heating the precursor material using the bubbler. Herein, the vaporization is preferably performed at a temperature between 50° C. and 116° C. More preferably, the precursor material is vaporized between 60° C. and 70° C. and is plasma-deposited on the substrate.

The precursor material is injected into the plasma reactor by a carrier gas. Herein, the carrier gas may be argon (Ar), nitrogen (N₂), helium (He) or hydrogen (H₂). Preferably, the carrier gas may be argon (Ar). The reactive gas is preferably introduced into the plasma reactor at a flow rate of 10-100 sccm.

The temperature of the substrate in the plasma reactor is preferably room temperature. The internal pressure of the plasma reactor may be in the range from 10 mTorr to several Torr and is preferably 500 mTorr.

When the precursor-derived material is deposited using plasma as described above, a plasma polymer layer will be formed uniformly on the substrate, and cells will not adhere to the surface of the plasma polymer layer.

(3) A Step of Placing Shadow Mask Having a Predetermined Pattern on Plasma Polymer Layer

As used herein, the term “shadow mask” refers to a thin metal plate having formed therethrough small holes that can expose desired specific portions. For example, holes may be formed through the shadow mask to form predetermined line- or bar-like patterns when viewed from the top, and the distance between these patterns may be 100-500 μm.

The shadow mask may be made of any material and may have any shape, as long as it can be placed on the plasma polymer layer and a predetermined pattern can be formed thereon. Also, the material and shape of the shadow mask are not specifically limited. In the present invention, the predetermined pattern of the shadow mask has a plurality of holes which form a bar-like pattern when viewed from the top, and the distance between the holes may be 100-500 μm.

(4) A Step of Treating the Substrate, Having Shadow Mask Placed Thereon, with Reactive Gas Using Plasma

The step of treating the substrate with the reactive gas using plasma is a step of modifying the surface of the plasma polymer layer which is exposed to the reactive gas through the holes forming the patterns on the substrate having the shadow mask placed thereon, so as to have the property of promoting cell adsorption.

Preferably, the plasma treatment may be performed using a device for an inductively coupled plasma chemical vapor deposition. In the device for an inductively coupled plasma chemical vapor deposition, the substrate is placed such that it is not influenced by an electric field generated by an RF-inducing antenna disposed outside a reactor, while plasma can be formed near the substrate. Thus, damage to substrate by plasma is insignificant, and an efficiency of the device can be increased. In addition, various kinds of reactive gases can interact with each other to modify the surface and thus it is important that the reactive gases are injected uniformly. Also, the surface modification can be performed at a relatively low temperature.

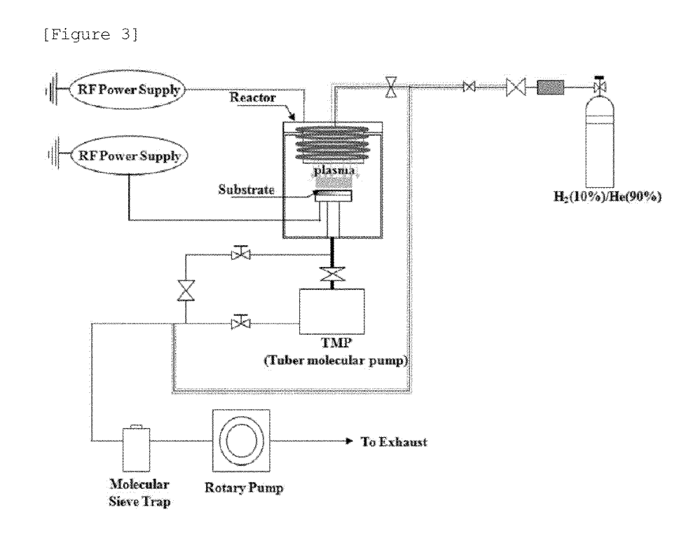

The device for an inductively coupled plasma chemical vapor deposition that is used to perform the step (4) will now be described in detail with reference to FIG. 3. FIG. 3 schematically shows the device for an inductively coupled plasma chemical vapor deposition that is used to fabricate a patterned substrate for a cell culture according to the present invention.

Referring to FIG. 3, the device for an inductively coupled plasma chemical vapor deposition generally comprises: a plasma reactor configured to form plasma therein; a vacuum unit including a vacuum pump (rotary pump) configured to control the internal pressure of the plasma reactor; and a power supply unit configured to apply voltage to an external electrode and internal electrode which are respectively disposed upper part of the plasma reactor and in the plasma reactor. The plasma reactor includes a substrate holder and an internal electrode disposed beneath the substrate holder.

Meanwhile, the external electrode and the internal electrode may be made of any material and may have any shape, like electrodes that are used in typical devices for plasma enhanced chemical vapor deposition. However, the external electrode preferably has a flat circular coil shape, and the internal electrode is preferably made of a material, which does not chemically react and is less contaminated. More preferably, the internal electrode is made of stainless steel.

The step of treating the substrate with the reactive gas using the device for an inductively coupled plasma chemical vapor deposition will now described in detail. The substrate having the plasma polymer layer formed thereon is placed on the substrate holder in the plasma reactor. And, a shadow mask having a predetermined pattern is fixed onto the substrate having the plasma polymer layer formed thereon. Then, the internal pressure of the plasma reactor is lowered to several mTorr close to a vacuum state by the vacuum pump (rotary pump), and in this state, the reactive gas is injected into the plasma reactor through the gas injection unit and a shower ring. When a voltage is applied to the external electrode and the internal electrode from the power supply unit in this state, reactive gas plasma generated by the external electrode and the internal electrode is formed between the plasma polymer layer-deposited substrate and the plasma reactor. The generated plasma can modify the properties of the surface of the exposed region, generated by the pattern of shadow mask, of the substrate having the shadow mask placed thereon.

One of the external electrode and the internal electrode may be used alone, but the use of both the external electrode and the internal electrode is more efficient and effective for formation of the patterned substrate for a cell culture. In addition, power that is applied to the external electrode as a power from the power supply unit of the plasma reactor may be 50-500 W, and preferably 70-100 W. In addition, treatment for modifying the surface may be performed for 30 seconds to 30 minutes, and preferably 1 minute.

The reactive gas that is used above may be any gas that can modify the surface of the plasma polymer layer so as to have the property of easily adsorbing cells. For example, the reactive gas may be a mixed gas of hydrogen and helium. If a mixed gas of hydrogen and helium is used as the reactive gas, the mixing ratio between hydrogen and helium may be 1:1-1:9.

(5) A Step of Removing the Shadow Mask from Substrate

After completion of the plasma treatment process, the shadow mask is removed and separated from the substrate, thereby fabricating a patterned substrate for a cell culture. Removal of the shadow mask from the substrate may be performed using any method that can achieve separation of the shadow mask from the substrate. In the present invention, the shadow mask is removed physically.

When the shadow mask is removed from the substrate, a patterned substrate having a predetermined pattern can be obtained, because the effect of reactive gas treatment after placing the shadow mask on surface of the substrate appears according to the pattern of the shadow mask.

Specifically, the exposed portion of the surface of the plasma polymer layer formed in the step (2) according to the pattern of the shadow mask is modified by treatment with the reactive gas using plasma so as to have the property of easily adsorbing cells. The plasma polymer layer portion covered by the shadow mask is blocked from the effect of treating with the reactive gas by the shadow mask, and thus when the shadow mask is removed, the plasma polymer layer as that formed in the step (2) is presented.

The substrate fabricated by the above-described method shows the property of adsorbing cells in portion of its surface with the predetermined pattern, and the property of inhibiting cell adsorption in the remaining portion of the surface. Thus, the environment for cells to adhere and be cultured can be provided only at the patterned portion. Thus, a patterned substrate for a cell culture can be fabricated according to the above-described method.

The present invention also provides a method for a cell culture with a pattern, comprising the step of culturing cells on the patterned substrate for the cell culture fabricated by the above method.

Cells that are used in the present invention are not specifically limited and may be, for example, cells isolated or activated from liver, kidney, spleen, bone, bone marrow, thymus, heart, muscle, lung, brain, testicle, ovary, islet, intestinal organs, ear, skin, gall tissue, prostate, bladder, embryo, the immune system, or the hematopoietic system. Preferably, the cells may be selected from the group consisting of microbial cells, animal cells, plant cells, animal organs, plant organs, neural cells, and vascular endothelial cells. More preferably, the cells may be vascular endothelial cells.

The present invention also provides a patterned cell chip having cells cultured on the patterned substrate for cell culture fabricated according to the above-described method.

As used herein, the term “cell chip” refers to a biochip capable of detecting complex physiological signals caused by the response of cells. The kind of cells cultured on the cell chip is as described above.

The present invention also provides a method of screening a material having an activity of inducing or promoting angiogenesis using the patterned cell chip.

As used herein, the term “angiogenesis” means the formation of new blood vessels by cells sprouting from the existing blood vessels. Angiogenesis is a physiological phenomenon that is involved in development or differentiation in the embryonic stage, women's menstrual cycle, wound healing, etc. Angiogenesis is involved in physiological phenomena called “angiogenic diseases”, including solid tumors, diabetic retinopathy, chronic rheumatoid arthritis, or arteriosclerosis, and it is a main therapeutic target to inhibit the proliferation of endothelial cells in such diseases and disorders.

When the patterned cell chip having cells cultured thereon or the present invention is treated with a test material suspected of inducing or promoting angiogenesis or considered to have potential to induce or promote angiogenesis, and then the proliferation pattern of cells thereon is analyzed, whether the test material has angiogenic activity can be determined in a simple and rapid manner.

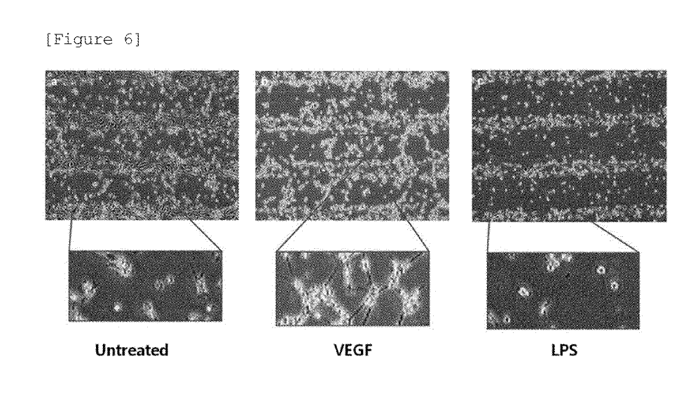

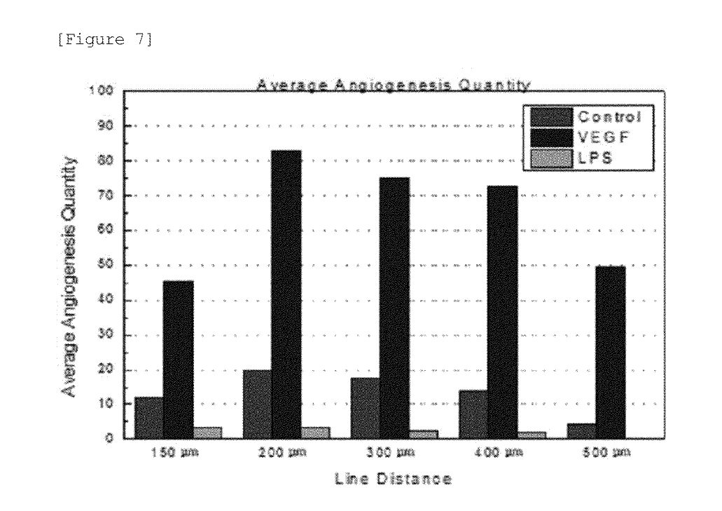

In an example of the present invention, a patterned cell chip was fabricated, and then untreated and treated with LPS or VEGF, and as a result, it could be seen that the group treated with LPS having the effect of inhibiting angiogenesis showed a significant decrease in the number of cells compared to the untreated group, whereas in the VEGF-treated group, the proliferation of cells significantly increased compared to that in the untreated group, and tube-shaped blood vessels were formed in the empted space of pattern (FIGS. 6 and 7). Thus, it can be seen that the cell chip according to the present invention makes it possible to perform an angiogenesis assay in a rapid and convenient manner compared to a conventional tube formation method.

Hereinafter, the present invention will be described in further detail with reference to examples. It is to be understood, however, that these examples are for illustrative purposes only and are not to intend to limit the scope of the present invention.

### Examples

### Example 1

### Fabrication of a Patterned Substrate for a Cell Culture

1-1. Formation of Plasma Polymer Layer on a Substrate

A thin layer was deposited on a substrate in a system for plasma enhanced chemical vapor deposition using hexamethyldisiloxane as a precursor material. Herein, the substrate was a glass slide having a size of 38 mm×75 mm. The substrate was washed, and then placed in the plasma reactor, and a thin layer of plasma polymerized hexamethyldisiloxane (PPHMDSO) was deposited on the substrate.

Specifically, the deposition was performed using the system for plasma enhanced chemical vapor deposition shown in FIG. 2. The precursor used was hexamethyldisiloxane (HMDSO), and the glass substrate used was a glass slide (Corning 2947) having a size of 75×38 mm and a thickness of 0.96-1.06 mm. The glass substrate was placed on a substrate holder in the PE-CVD reactor. HMDSO in the bubbler was vaporized by heating it at 61° C., and the basic pressure in the reactor was lowered to several mTorr using the rotary pump. Then, vaporized HMDSO was transferred into the reactor using 10 sccm of Ar (99.99%) gas as a bubbling gas. For generation of RF (radio-frequency) plasma, plasma was generated by applying a substrate bias plasma power of 10 W using a plasma generator connected to a matching box, thereby fabricating a substrate having a PPHMDSO thin layer. Herein, the RF plasma power had a frequency of 13.56 MHz, and the inside of the reactor was maintained at a constant pressure of 500 mTorr during deposition. In this way, a substrate having a plasma polymer layer formed thereon, that is, a substrate comprising a glass slide having a plasma polymerized hexamethyldisiloxane thin layer formed thereon, was fabricated.

1-2. Fixing of Patterned Shadow Mask, Followed by Treatment with Reactive Gas Using Plasma

To the substrate having PPHMDSO-deposited thereon by the method of Example 1-1, a metal shadow mask having a predetermined pattern was fixed. The used shadow mask had a distance of 300-400 μm between the patterns. The substrate having the shadow mask fixed thereto was placed in a plasma reactor, and the internal pressure of the reactor was maintained to a vacuum of several mTorr by a rotary pump. Then, a reactive gas consisting of a mixed gas of hydrogen (10%)/helium (90%) was introduced into the reactor through a shower ring at a flow rate of 10 sccm using a mass flow controller (MFC) and was treated with plasma at an inductively coupled plasma (ICP) power of 100 W and a pressure of 200 mTorr for 1 minutes, thereby modifying the surface of the substrate. Then, the shadow mask was removed and separated from the substrate, thereby fabricating a patterned substrate for cell culture.

1-3. Examination of a Cell Culture with a Pattern

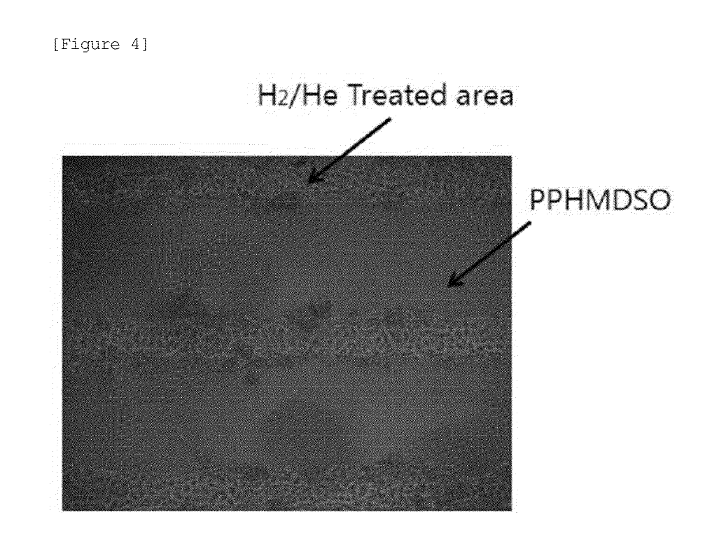

Endothelial cells were cultured on the patterned substrate fabricated in Example 1-2, and the results of the culture are shown in FIG. 4. As shown in FIG. 4, in the PPHMDSO area whose surface was not modified due to the shadow mask, the adsorption of the cells was inhibited, and in the patterned area modified by treatment with the reactive gas, the adsorption and proliferation of the cells occurred. Thus, it was confirmed that it is possible to fabricate a patterned cell chip on which cells are cultured while forming a specific pattern.

### Test Example 1

### Angiogenesis Assay Using a Patterned Substrate for the Cell Culture Fabricated According to the Present Invention

In an angiogenesis assay using the fabricated substrate, bovine aortic endothelial cells (BAECs) were used. To prevent contamination, the substrate fabricated in Example 1-2 was placed in a Petri dish and irradiated with UV light for 16 hours prior to the test.

BAEC cells were seeded in 2 ml of complete medium (low glucose DMEM (Dulbecco's Modified Eagle's Medium, WELGENE) containing 1× penicillin/streptomycine (WELGENE) and 20% fetal bovine serum (FBS, WELGENE)) at a cell concentration of 2.5×10⁵ cells/ml, and then cultured in a 5% CO₂ incubator at 37° C. for 6 hours. Next, the complete medium was replaced with the following media.
untreated plate (normal control group): 10 ml of low-glucose DMEM containing 1× penicillin/streptomycin and 0.2% FBS;
LPS (lipopolysaccharide)-treated plate (negative control group): 10 ml of low-glucose DMEM containing 1× penicillin/streptomycin, 0.2% FBS and 100 ng/ml of LPS; or
VEGF (vascular endothelial growth factor)-treated plate (positive control group): 10 ml of low-glucose DMEM containing 1× penicillin/streptomycin, 0.2% FBS and 100 ng/ml of VEGF.

To prevent cells on the substrate from being dried due to evaporation of the media in the incubator, the volume of the media was increased to 10 ml, and to more accurately confirm the effect of the positive control group (VEGF-treated plate), starvation basic medium having a decreased FBS (a kind of growth factor) concentration of 0.2% was used. After replacing the medium, the cells were cultured in a 5% CO₂ incubator at 37° C. for 18 hours, and angiogenesis that occurred on the substrate was observed with an optical microscope. The results of the observation are shown in FIGS. 6 and 7.

As a result, as shown in FIG. 6, the culture patterns of the cells did differ among the three conditions (untreated, VEGF-treated, and LPS-treated). In comparison with the untreated case, in the case treated with LPS, angiogenesis was inhibited while a smaller number of cells could be observed in the regions between cell adsorbing H₂/He plasma treated areas, but in the case treated with VEGF, angiogenesis was induced and promoted, therefore a significantly larger number of the cells were observed in the regions between cell adsorbing H₂/He plasma treated areas, and tube-shaped blood vessels were newly formed. In addition, as shown in FIG. 7, the average quantity of angiogenesis did significantly differ among the three conditions. This suggests that the patterned cell chip according to the present invention can be used as a substrate for performing an angiogenesis assay.

As described above, the method for fabricating a patterned substrate for a cell culture according to the present invention is a fabrication method forming pattern with a region adsorbing cells thereon and the other region inhibiting cell adsorption and making those regions distinguishable from each other by using a precursor, a shadow mask and plasma treatment with reactive gas. The fabrication method is simple and makes it possible to culture cells only in a desired region on the fabricated substrate, and thus it can be applied to various cell chips.

In addition, a cell chip having cells cultured on the substrate fabricated according to the above-described method can be used for an angiogenesis assay and makes it possible to analyze the angiogenic activity of a test material in a convenient manner. Thus, the use of the patterned cell chip can screen a material that promotes or induces angiogenesis.

1. A method for fabricating a patterned substrate for a cell culture, comprising the steps of:
(1) preparing a substrate;
(2) depositing a plasma polymer layer by using a precursor material on the substrate;
(3) placing a shadow mask having a predetermined pattern on the plasma polymer layer;
(4) treating the substrate, having the shadow mask placed thereon, with a reactive gas using plasma; and
(5) removing the shadow mask from the substrate.

2. The method for fabricating a patterned substrate for the cell culture of claim 1, wherein the substrate is made of a material selected from the group consisting of glass, plastic, metal and silicon.

3. The method for fabricating a patterned substrate for the cell culture of claim 1, wherein the precursor material is one or more selected from the group of siloxane-based compounds consisting of hexamethyldisiloxane, octamethyltrisiloxane, decamethyltetrasiloxane, hexamethylcyclotrisiloxane, octamethylcyclotetrasiloxane, and decamethylcyclopentasiloxane.

4. The method for fabricating a patterned substrate for the cell culture of claim 1, wherein the plasma polymer layer in step (2) is deposited by using plasma enhanced chemical vapor deposition (PE-CVD).

5. The method for fabricating a patterned substrate for the cell culture of claim 4, wherein the plasma enhanced chemical vapor deposition is performed at a power of 5-20 W.

6. The method for fabricating a patterned substrate for the cell culture of claim 1, wherein the shadow mask having the predetermined pattern in step (3) has a distance of 100-500 μm fall between patterns.

7. The method for fabricating a patterned substrate for the cell culture of claim 1, wherein treating the substrate with the reactive gas in step (4) is performed by using inductively coupled plasma chemical vapor deposition (ICP-CVD).

8. The method for fabricating a patterned substrate for the cell culture of claim 1, wherein the reactive gas is a mixed gas of hydrogen and helium.

9. The method for fabricating a patterned substrate for the cell culture of claim 8, wherein the mixing ratio of hydrogen and helium is 1:1-1:9.

10. The method for fabricating a patterned substrate for the cell culture of claim 7, wherein the inductively coupled plasma chemical vapor deposition is performed at a power of 50-500 W.

11. The method for fabricating a patterned substrate for the cell culture of claim 7, wherein the inductively coupled plasma chemical vapor deposition is performed for 30 seconds to 30 minutes.

12. A patterned substrate for a cell culture fabricated by the method of claim 1.

13. A method for a cell culture with a pattern, comprising the steps of:
fabricating a patterned substrate for the cell culture according to the method of claim 1; and
culturing cells on the patterned substrate for the cell culture.

14. The method for the cell culture with a pattern of claim 13, wherein the cell is selected from the group consisting of microbial cells, animal cells, plant cells, animal organs, plant organs, neural cells, and vascular endothelial cells.

15. A patterned cell chip having cells cultured on the patterned substrate for cell culture fabricated by the method of claim 1.

16. The patterned cell chip of claim 15, wherein the cells are selected from the group consisting of microbial cells, animal cells, plant cells, animal organs, plant organs, neural cells, and blood vessel cells.

17. A method of screening a material having an activity of inducing or promoting angiogenesis using the patterned cell chip of claim 15.
